(19) United States
(12) Patent Application Publication
(10) Pub. No.: US 2018/0181709 A1
(43) Pub. Date: Jun. 28, 2018

(54) The Use of Bioinformatic Data in Autologous Cell Therapies

(71) Applicant: GE HEALTHCARE UK LIMITED, BUCKINGHAMSHIRE (GB)

(72) Inventors: Mark Samuel Jonathan Briggs, Cardiff Wales (GB); Nicholas Thomas, Cardiff Wales (GB)

(21) Appl. No.: 15/738,397

(22) PCT Filed: Jun. 29, 2016
(86) PCT No.: PCT/EP2016/065093

§ 371 Date: Dec. 20, 2017

(30) Foreign Application Priority Data
Jun. 30, 2015 (GB) 1511419.2

(51) Int. Cl. G06F 19/24; G06F 19/18; G16H 50/20

(52) U.S. Cl. CPC G06F 19/24; G16H 50/20; G06F 19/18

(57) ABSTRACT

Disclosed is a method for optimising an ex-vivo autologous cell culture procedure, said method including the steps of: obtaining and storing a patient's bioinformatic data; comparing said patient's bioinformatic data with known data in the form of bioinformatic data collected from other patients and/or other predetermined data such as genomic or proteomic data; and selecting ex-vivo cell culture procedure parameters based on the comparison between said patient's bioinformatic data and said known data. The selection can also be influenced by using the better data and/or culture parameter indicators determined by monitoring the outcome of plural cellular therapy attempts.

### FIELD OF THE INVENTION

The present invention relates to the use of bioinformatic data associated with a patient to provide data relating to autologous cell therapy techniques for the patient. Various uses of such data include optimising ex-vivo cell culture systems; selecting the most suitable treatment from a predetermined treatment list; and feedback of patient outcomes to improve the accuracy of the therapy techniques.

### BACKGROUND OF THE INVENTION

Personalised cell therapies have shown significant promise latterly. These therapies, in principal, involve taking a cell sample from a patient, separating the cells of interest, for example T cells or stem cells, optionally modifying such cells, multiplying the optionally modified cells, and administering the multiplied cells to the patient. As cells from each patient must be processed in isolation, conventional batch manufacturing practices common to nearly every medical production industry cannot be employed in autologous therapies. Thus, whilst the medical results shown significant promise, the commercialisation of ‘personalised’ cell therapy remains a significant challenge due the lack of understanding concerning optimising the unique batch manufacturing approach, the lack of clinical understanding of predictive outcomes, e.g. potency of the multiplied cells, as well as a lack of economies of scale presently, making the personalised approach prohibitive expensive.

It has been observed by the inventors that the inherently variable starting materials (quantity and quality of starting biological material) present challenges in ex-vivo multiplication of cells and automated manufacturing, and leads to a concomitant impact upon cell growth, and undue expense. Whilst applicable to all cell sources and therapies these challenges are particularly pertinent to autologous therapies whereby each administered cell dose is unique to the recipient.

### SUMMARY OF THE INVENTION

This disclosure describes the application of bioinformatics to ‘triage’ incoming patient samples (pertinent biological material such as tissue, cells etc.) and to allocate the samples to a manipulation, processing and expansion regimen that maximises both the utilisation of manufacturing resources and positive clinical outcome i.e. a better end product quality and potency. In one embodiment it is envisaged that software can use known and predicted properties of starting biological material, based on bioinformatic data, to assign a suitable processing workflow to result in strict inventory control (pre-allocation of equipment, consumables, reagents) and scheduled manufacturing slots. This will reduce the need for dynamic, or ad hoc modification, of standard protocols in response to individual sample status, such as extension of culture time or addition of extra growth factors/media, with reduction in material wastage (dedicated & limited shelf-life materials prepared ‘just in case required’), labour costs (underutilised staff & overtime costs) and minimise production line (equipment) dead time (underutilisation).

It is envisaged also that bioinformatic data will also to support clinical decisions in the selection of a treatment programme with the highest probability of success on an individual basis.

With the broader application of cell therapy treatments and the ability to track outcomes, it is envisaged also that outcome data can be used to add to the a predictive data set, such that the quality of the data will improve, which in turn will increase the accuracy and utility of the predictive analysis.

Embodiments of the invention provide a method for optimising an ex-vivo cell culture procedure, said method including the following steps, in any suitable order:

i) obtaining and storing a patient's bioinformatic data;

ii) comparing said patient's bioinformatic data with known data, in the form of data collected from other patients and/or other predetermined data such as genomic or proteomic data; and

iii) selecting ex-vivo cell culture procedure parameters based on the comparison between said patient's bioinformatic data and said known data.

Herein, bioinformatic data includes, but is not limited to, one or more of: sex; age; weight; BMI; diet; ethnicity; patient health indictors such as patient current medical condition, medical history, family medical history; specific patient sample related data such as cell multiplication rate, cell count, cell immune response, diagnostic indicators such as the presence of genetic or protein biomarkers; data from diagnostic tests; data from DNA, RNA and/or protein analysis of blood or other tissues.

Other aspects of the invention are set out in the claims and are described below.

The invention extends to any combination of features disclosed herein, whether or not such a combination is mentioned explicitly herein. Further, where two or more features are mentioned in combination, it is intended that such features may be claimed separately without extending the scope of the invention.

### BRIEF DESCRIPTION OF THE DRAWINGS

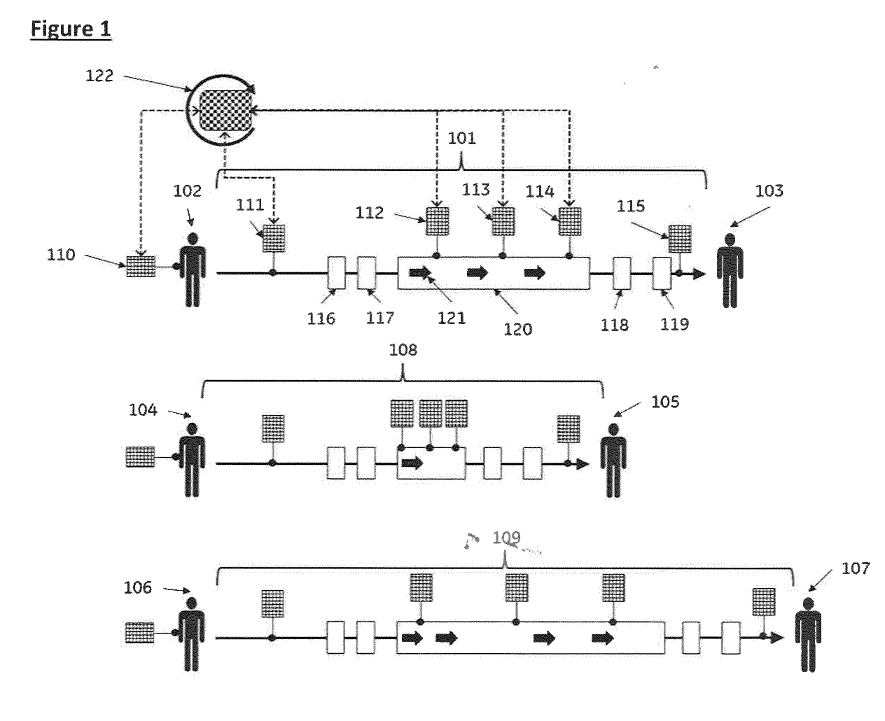

The invention can be put into effect in numerous ways, illustrative embodiments of which are described below with reference to the drawings, wherein FIG. 1 shows a schematic representation of a cell culture system operated in accordance with a method, all as described below.

The invention, together with its objects and the advantages thereof, may be understood better by reference to the following description taken in conjunction with the accompanying drawings.

Referring to FIG. 1 an autologous cell therapy workflow [101] is illustrated schematically, which comprises a series of fixed and variable operations, described in more detail below, for processing cells derived from a patient [102] to a state suitable for administration after processing to the same patient [103] as a cell therapy. Due to natural or disease based inherent biological variations between individual patients and their cells to be processed from patient samples to therapeutic products the processing workflow [101] may need to varied, and such variant workflows are shown in variants [108 & 109] which allow modification of certain aspects of the workflow to accommodate such biological variations in the patient's cells, e.g. different growth rates in culture, and therefor permit the optimum processing of cells derived from different patients [104 & 106] to individual cell therapies for administration to the same respective donors [105 & 107].

Within the workflow [101] certain elements which relate to physical processes, e.g. cell purification and cell concentration by centrifugation, filtration or other means, are invariant [116, 117, 118 & 119] as these processes may be performed using fixed procedures which do not need to take account of biological variations and behaviours between processed cells derived from different patients. The workflow may also comprise one or more variable processes, e.g. cell culture and expansion [120], which may be highly dependent on the biological characteristics of the cells in individual patient samples and consequently require different operations within the variable process, e.g. media changes or media perfusion [121], to be carried out at different frequencies, in different volumes or as other variants to optimise the processing of individual patient samples. The required duration of such variable processes may also alter between different patient samples to account for different cell growth rates and/or other biological variances. Process variations may be applied on an ad-hoc basis in response to the behaviour of cells being processed, e.g. shortening or lengthening the cell culture and expansion process duration to account for cells which are growing faster or shorter that the norm.

Such ad-hoc variations cannot be accommodated within a standard regulated facility performing the processing of many patient's samples in parallel as they may have significant adverse impact on;

a) the ability of a facility to provide optimum individual patient care at the time it is required or to appropriately schedule processing of multiple patient samples in line with the demands of ongoing treatment regimens;

b) the efficiency of the facility where variable operations subject to ad-hoc variances make scheduling of equipment use difficult, may lead to sub-optimum use of capital equipment and reagents, and other factors which raise the costs of providing cell therapies and reduce the capability of the facility to provide optimum processing of patient samples, and;

c) the ability of the facility to carry out operations in accordance with regulated procedures where ad-hoc variations and deviations from standard operating procedures conflict with regulatory GMP requirements for fixed and invariant procedures.

The method of the present invention seeks to overcome these issues by providing means to use pre-defined and standardised variations in the cell processing workflow in accordance with triaging of patients and patient samples using bioinformatics analysis of patient and patient sample data. Patient data [110] may include, but are not limited to, a patient's age and sex, current medical condition, medical history, data from diagnostic tests, data from genomic DNA sequencing and data from RNA and/or protein analysis of blood or other tissues. Sample data [111] acquired from analysis of the patient sample may include, but are not limited to, data from analysis of cell surface protein marker expression by flow cytometry or imaging, analysis of RNA and/or microRNA expression by RT-PCR or microarray hybridisation, biochemical, metabolic or respirometry analyses and DNA sequencing.

Bioinformatics analysis of collective patient [101] and sample [111] data is used to generate a prediction of the likely behaviour of a given patient's cells during processing and enable the selection of an appropriate pre-defined processing workflow variant [101, 108 & 109] to provide optimum processing of the patient sample to a therapeutic preparation within a regulated environment. Workflow variations selected using such bioinformatics analysis may include, but are not limited to, increased or decreased cell culture and expansion times, use of different culture media, different culture volumes and/or batch feeding or media perfusion protocols, additions of culture supplements and other means to control cell concentrations and/or growth rates in culture.

Patient data [110] and sample data [111] may be supplemented with data acquired at different stages in the cell processing workflow [112, 113 & 114] and from the processed therapeutic product at the end of the workflow prior to administration to the donor patient [115]. Such supplemental process and product data may comprise, but are not limited to, cell counts, cell viability, physicochemical data (e.g. cell culture pH, oxygen content and consumption, and metabolite concentrations) and cell phenotype and genotype analysis. Phenotype analysis may comprise measurement of protein, RNA or other markers specific to certain cell types to determine the relative abundance of a desired therapeutic cell type at different stages in the processing workflow. Genotype analysis may comprise DNA sequencing, DNA profiling, karyotype analysis or other means to monitor the genetic stability and/or integrity of the processed cells through the processing workflow.

To enable the continual improvement of the predictive utility of bioinformatics analysis of patient [110], sample [111], process [112, 113 & 114] and product [115] data for selection of variant workflows [101, 108 & 109] all data is accumulated in a database [122]. Constant iterative analysis of data accumulated within the database [122] over time from successive processing of multiple patient samples using variant workflows [101, 108 & 109] is used to refine the selection process for the most appropriate variant workflow based on the patient [110] and sample [111] data. Such iterative analysis may be used to establish the most predictive parameter(s) within the patient and/or sample data providing the optimum selection of a workflow variant for efficient processing of any given patient sample to a therapeutic product.

Such iterative data analysis may include, but is not limited to, pair-wise correlation analysis of all parameters in process [112, 113 & 114] and product [115] data with patient [110] and sample [111] data. Correlation analysis may be used to establish that certain patient and/or sample parameters show a high degree of correlation with process and/or product parameters and are therefore suitable for use in selecting an optimum processing variant while other parameters do not show correlation and are consequently not suitable for use in determining choice of processing variant. For example it may be found that there is a good inverse correlation between cell culture expansion rate measured from process data [112, 113 & 114] and patient age derived from patient data [110]. In such a case where cell growth rates are found to be inversely correlated with patient age it would be appropriate to select a process variant with a short cell expansion phase [108] for younger patients and a process variant with a longer cell expansion phase [109] for processing cells from an elderly patient. Similarly if it were found that low or high abundance of a certain cell type in the patient sample data [111] showed good correlation with a requirement for extended or shortened cell expansion this parameter may be used in conjunction with other patient and/or sample data to select an optimum variant of the cell processing workflow.

Conversely if a parameter in the patient and/or sample data shows no correlation with process or product data such a parameter may be removed from those used to select an optimum processing variant. For example it may be determined in the course of iterative analysis of successive processing of many patient samples that there is no correlation between patients' sex and cell expansion rates in culture and therefore this information in the patient data would not be used to determine the choice of processing variant.

Ongoing iterative data analysis also permits modification of the nature of patient [110], sample [111], process [112, 113 & 114] and product [115] data. Where data parameters collected at one or more of these points are found to have no predictive value in selection of a variant processing workflow appropriate to the patient sample, collection of such parameters may be discontinued. Where new parameters become available through discovery of new analytes or biomarkers, or through application of new analytical procedures, such parameters may be added to patient, sample, process or product data and the predictive value of the new parameters assessed in combination with existing parameters. Such evolution will increase the predictivity of the collective data and remove costs associated with ongoing acquisition of redundant data.

Other suitable means for data analysis include principal component analysis or data clustering techniques including, but not limited to, K-means clustering, hierarchical clustering and self-organising map (SOM) analysis. Such analyses provide means to reduce the complexity of multi-variate data and to identify combinations of data parameters which in concert provide means to select an optimum processing variant for any given patient.

Ongoing collection and analysis of patient, sample, process and product data therefore provides a constantly improving means to select an optimum standardised pre-defined workflow for each patient sample processed within a facility. Such selection removes the need for ad-hoc process variations based on operator judgement and allows a defined collection of regulatory approved workflows to be scheduled and implemented providing optimum therapeutic and cost efficiencies.

### EXAMPLE

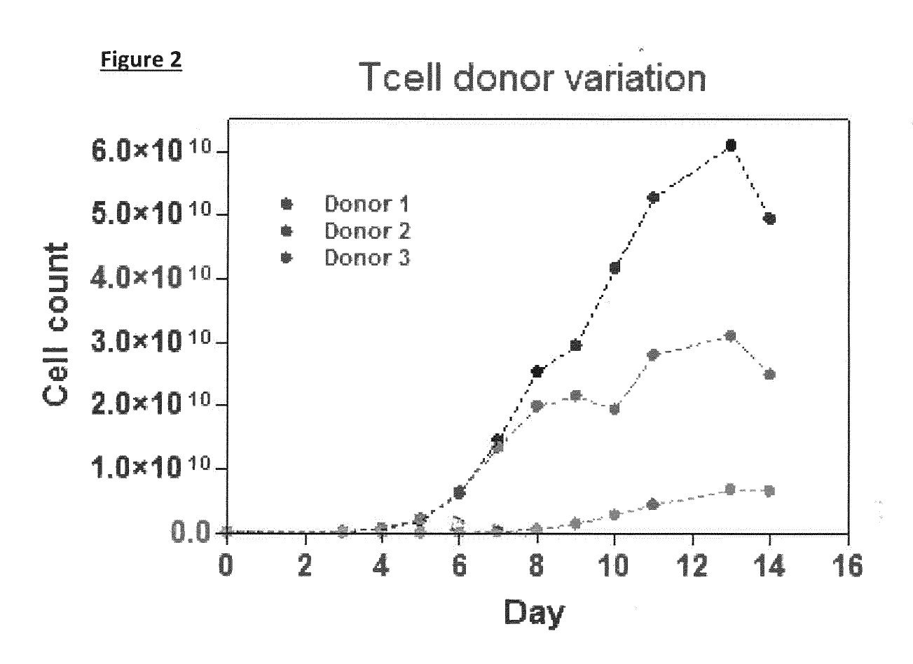

Samples of blood were taken from three donors and their T cells extracted. Approximately equal numbers of seed cells from each donor were each cultured in the same manner in a small scale bioreactor for 14 days using known techniques, and the cell density for each donor's cells was monitored daily as culturing progressed. The results are tabulated in FIG. 2, and show significant variation in cell count between the three donors over time. This indicates that bioinformatic data can play a significant role in determining the optimal regime for cell culture.

Further, where a minimum number of cells are required for therapy, it would be possible to increase the rate at which known growth factors are added to the culture media, in order to reduce the time taken to produce the desired number of cells. Bioinformatic data, for example initial in vitro cell multiplication rates, can be used to predict the cell culture multiplication rate of a patient's cells.

Although one embodiment of the invention has been described and illustrated, it will be apparent to the skilled addressee that additions, omissions and modifications are possible without departing from the scope of the invention claimed.

1. A method for optimising an ex-vivo autologous cell culture procedure, said method comprising the following steps, in any suitable order:
i) obtaining and storing a patient's bioinformatic data;
ii) comparing said patient's bioinformatic data with known data, in the form of bioinformatic data collected from other patients and/or other predetermined data such as the patient's genomic or proteomic data; and
iii) selecting ex-vivo cell culture procedure parameters based on the comparison between said patient's bioinformatic data and said known data.

2. A method as claimed in claim 1, wherein said patient's bioinformatic data and said bioinformatic data collected from other patients includes one or more of: sex; age; weight; BMI; diet; ethnicity; patient health indictors such as patient current medical condition, medical history, family medical history; specific patient sample related data such as cell multiplication rate, cell count, cell immune response, diagnostic indicators such as the presence of genetic or protein biomarkers; data from diagnostic tests; data from DNA, RNA and/or protein analysis of blood or other tissues.

3. A method as claimed in claim 1, wherein said cell culture parameters include: period of culture; the ratio of constituents of cell culture media; rate of additional media added to the culture (dilution rate); rate at which effluent is removed from the culture, culture filtration regimen volume of culture.

4. A method as claimed in claim 1, wherein cell numbers are counted during culture, and one or more of the parameters, for example the concentration of growth factor, is/are altered according said count.

5. A method as claimed in claim 1 further including the step of monitoring plural outcomes of cellular therapy based on cells cultured according to said selected parameters, and determining which of the patients' bioinformatic data and/or selected parameters provides the better indicator(s) of a successful therapy outcome, and using said better indicator(s) to further influence said selection of said parameters.

6. A method as claimed in claim 5, wherein said patients' bioinformatic data providing a better indicator of a successful outcome is cell multiplication rate, and/or said cell culture parameter providing a better indicator of a successful outcome is the period of culture.

7. A method as claimed in claim 5, wherein said comparison step provides a probability of therapy efficacy.

8. An autologous cell culture system operated in accordance with a method according to claim 1.
